(19) United States
(12) Patent Application Publication
(10) Pub. No.: US 2026/0068861 A1
(43) Pub. Date: Mar. 12, 2026

(54) HABITAT FOR INSECTS

(71) Applicant: Nutrinsectos de México, S.A.P.I. de C.V., Guadalajara (MX)

(72) Inventor: Héctor Jiménez Franco, Guadalajara (MX)

(73) Assignee: Nutrinsectos de México, S.A.P.I. de C.V., Guadalajara (MX)

(21) Appl. No.: 19/318,825

(22) Filed: Sep. 4, 2025

Related U.S. Application Data
(60) Provisional application No. 63/691,564, filed on Sep. 6, 2024.

(51) Int. Cl. A01K 67/362; A01K 67/364; A01K 67/368

(52) U.S. Cl. CPC A01K 67/362; A01K 67/364; A01K 67/368

(57) ABSTRACT

Embodiments relate to a habitat for insects. The habitat includes a laminated structure made from plural sheets. Each sheet has grooves formed on either side of the sheet, wherein the grooves of one sheet are not parallel to the grooves of an adjacent sheet. Each sheet is made from cardboard and is coated with cellulose or other similar material.

### CROSS-REFERENCE TO RELATED APPLICATIONS

This patent application is related to and claims the benefit of priority to U.S. Provisional Ser. No. 63/691,564 , filed on Sep. 6, 2024, the entire contents of which is incorporated herein by reference.

### FIELD OF THE INVENTION

Embodiments can relate to a habitat for insects.

### BACKGROUND OF THE INVENTION

Insect agriculture has been practiced for centuries in Asian countries as part of their diets, though systematic documentation on cricket rearing is less prevalent. Commercial cricket rearing gained traction in the 20th century, particularly during the 1950s and 1960s in the United States and Europe, where crickets were bred for bait and pet food. Over the past 70 years, this practice has relied on egg crates and other grid-shaped cardboard structures as primary habitats. While these materials offer some benefits, they fall short in providing an ideal environment for a thriving cricket colony and ensuring maximum survival rates. The inventive habitat is directed at overcoming one of more of the above-mentioned problems.

### SUMMARY OF THE INVENTION

The inventor has identified key factors that enhance the likelihood of a thriving insect (e.g., cricket) colony, maximizing yield by ensuring maximum survival. The disclosed laminate structure represents a solution that not only offers the ideal habitat for crickets in captivity, but also serves as an optimal housing structure for crickets during transportation and retail display. This can be achieved through innovative material use, structural design, and multifunctionality that sets the inventive habitat apart from traditional methods. The inventive habitat is an innovative housing structure designed to provide optimal conditions for the mass rearing, transportation, and retail display of crickets and other insects. The technology can significantly enhance the survival rates and overall well-being of cricket colonies by introducing material applications, unique structural designs, and multifunctional capabilities.

Existing laminate block structures can be appreciated from evaporative cooling media/pads, which are available here: https://www.grainger.com/product/4KAZ9?gucid=N:N:PS:Paid:GGL:CSM-2295:4P7A1P:20501231&gad_source=1&gclid=CjwKCAjw8rW2BhAgEiwAoRO5rHbM-YigO-HlwCxl0iVHlloBayYPg7tGbNqLn6yXIghbqRuOdJjSxxoCYVIQAvD_BwE&gclsrc=aw.ds.

An exemplary embodiment relates to a habitat for insects. The habitat includes plural sheets. One sheet is arranged adjacent to another sheet. Each sheet has a surface ornamentation to generate plural abutment points and plural volumes of spaces between two adjacent sheets.

Each volume of space provides a living, breeding, and/or hiding space for an insect. The plural sheets form a structure having a top and a bottom. The plural volume of spaces forms a column defining a pathway from the top to the bottom. The pathway facilitates passage of frass within the column in a direction from the top to the bottom.

In some embodiments, the column and plurality of spaces are configured to facilitate providing plural living, breeding, and/or hiding spaces for an insect while also facilitating passage of frass within the column in a direction from the top to the bottom without disruption of the living, breeding, and/or hiding spaces.

In some embodiments, the plural sheets form plural columns, each column having plural volume of spaces.

In some embodiments, at least one sheet is made of cardboard.

In some embodiments, each sheet is made of cardboard.

In some embodiments, at least one sheet is made of non-recycled cardboard.

In some embodiments, each sheet is made of non-recycled cardboard.

In some embodiments, at least one sheet is made of cardboard coated with cellulose.

In some embodiments, each sheet is made of cardboard coated with cellulose.

In some embodiments, at least one sheet is adhered to an adjacent sheet via an adhesive.

In some embodiments, each sheet is adhered to an adjacent sheet via an adhesive.

An exemplary embodiment relates to a habitat for insects. The habitat includes a laminated structure comprising plural sheets. Each sheet has a first surface and a second surface. Each sheet includes an undulated profile having plural crests and plural throughs. For each sheet: an apex of each crest forms a ridge on the first surface; an apex of each ridge forms a ridge on the second surface; a volume of space between two adjacent crests forms a groove on the first surface; a volume of space between two adjacent throughs forms a groove on the second surface. The plural sheets include a first sheet having plural grooves and a second sheet having plural grooves. Each of the plural grooves of the first sheet has a longitudinal axis G₁. Each of the plural of grooves of the second sheet has a longitudinal axis G₂. The plural sheets are arranged in the laminated structure so that: the first sheet is adjacent the second sheet; a crest of the first sheet abuts a trough of the second sheet; and G₁ is not parallel to G₂.

An exemplary embodiment relates to a method of forming a habitat for insects. The method involves generating a laminated structure comprising plural sheets, each sheet having a first surface and a second surface, each sheet including an undulated profile having plural crests and plural throughs, the plural sheets include a first sheet having plural grooves and a second sheet having plural grooves. The method involves placing each sheet adjacent another sheet so that: an apex of each crest forms a ridge on the first surface; an apex of each ridge forms a ridge on the second surface; a volume of space between two adjacent crests forms a groove on the first surface; a volume of space between two adjacent throughs forms a groove on the second surface. Each of the plural grooves of the first sheet has a longitudinal axis G₁. Each of the plural of grooves of the second sheet has a longitudinal axis G₂. The first sheet is adjacent the second sheet. A crest of the first sheet abuts a trough of the second sheet. G₁ is not parallel to G₂.

In some embodiments the method involves forming each sheet from cardboard.

In some embodiments the method involves forming each sheet from non-recycled cardboard.

In some embodiments the method involves coating each sheet with cellulose.

An exemplary embodiment relates to a method of maintaining a habitat for insects. The method involves placing insects into a structure including plural columns, each column including plural volumes of spaces. The method involves allowing insects to live, breed, and hide in the plural volumes of spaces within the structure. The method involves placing food on a top of the structure. The method involves allowing insects to access the food via pathways defined by the plural columns. The method involves allowing frass to exit at a bottom of the structure.

In some embodiments the method involves cutting the structure into at least one block or sub-block.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIGS. 1-2 show an exemplary habitat for insects in the form of a cubic laminated structure.

FIGS. 3-4 show an exemplary habitat for insects with a sheet having a cut-away view.

FIG. 5 shows a close up front view of an exemplary laminated structure.

FIG. 6 shows a close up view of a segment of an exemplary habitat for insects demonstrating food being placed on a top portion of the habitat and frass exiting a bottom portion of the habitat.

FIG. 7 shows a perspective view of a segment of an exemplary habitat for insects illustrating formation of a plurality of columns.

FIG. 8 shows a close up perspective view of a top portion of an exemplary habitat for insects illustrating formation of a plurality of living spaces for insects.

FIG. 9 shows an exemplary method of forming a habitat for insects.

FIG. 10 shows an exemplary method of maintaining a habitat for insects.

FIG. 11 depicts a side view of an exemplary habitat showing the sequential layering of sheets.

FIG. 12 depicts a front view of an exemplary habitat.

FIG. 13 depicts an example of live insect interaction with an embodiment of the habitat.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

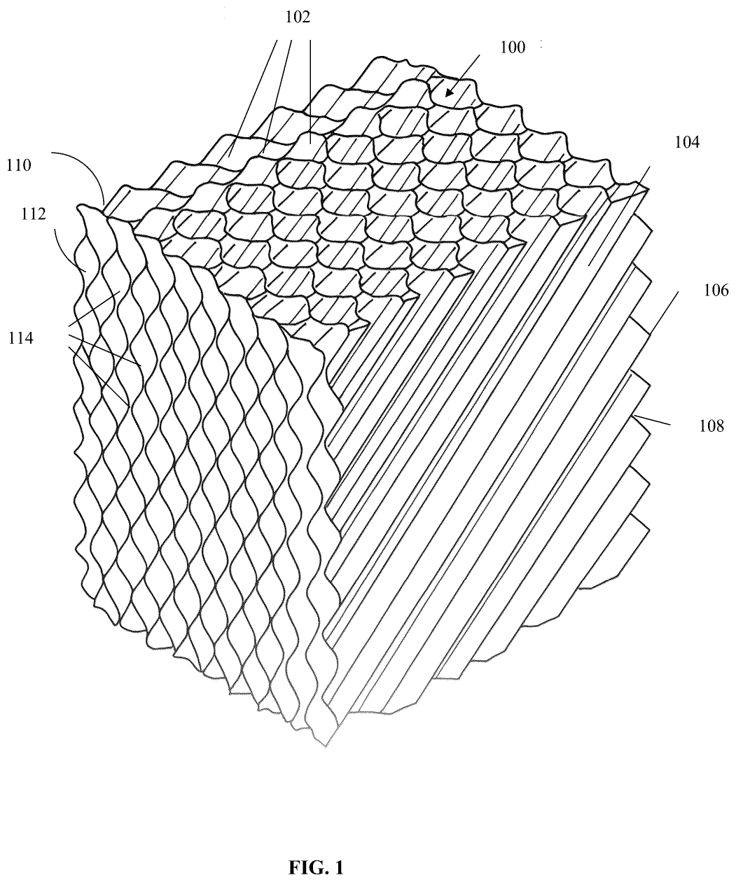

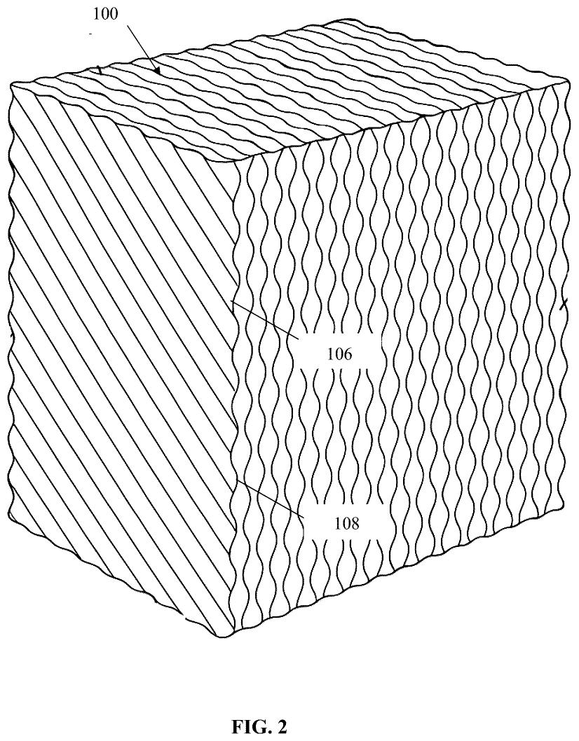

Referring to FIGS. 1-2, embodiments can relate to a habitat 100 for insects. The habitat 100 can be a for living, breeding, rearing, transportation, etc. of insects. While it is contemplated for the insects to be crickets, the habitat 100 can be used for other types of insects (e.g., cockroaches, grasshoppers, katydids, beetles, etc.). The habitat 100 can be a structure configured to house insects and provide shelter, resources, environment, etc. conducive for the insects to survive and reproduce.

The habitat 100 can include plural sheets 104. At least one sheet 104 of the plural sheets 104 can be arranged adjacent to another sheet 104. At least one sheet 104 can have a surface ornamentation that generates one or more abutment points and one or more volumes of spaces 116 between its adjacent sheet 104. At least one volume of space 116 can be configured to provide a living, breeding, and/or hiding space for an insect. It is contemplated for the plural sheets 104 to form a structure 102. The structure 102 can have a top 120 and a bottom 122. The structure 102 can be configured such that the volume of spaces 116 form one or more columns 118 (e.g., columnar pathways). One or more of the columns 118 can extend from the top 120 to the bottom 122—e.g., provide ingress and egress via the top 120 and bottom 122. It is contemplated for the plural volume of spaces 116 for each column 118 to define a pathway from the top 120 to the bottom 122. One or more of the pathways can facilitate passage of food and/or frass within the column 118 in a direction from the top 120 to the bottom 122. For instance, the insect can use the volume of space 116 as a living space to live, sleep, eat, etc. As the insect defecates, the fecal matter can move from the living space and fall (e.g., free fall or be pushed by an insect) towards the bottom 122 and exit the structure 102 via an egress opening of the column 118. As can be appreciated, the column(s) 118 and plurality of spaces 116 can be configured to facilitate providing plural living, breeding, and/or hiding spaces 116 for an insect while also facilitating passage of food and/or frass within the column(s) 118 in a direction from the top 120 to the bottom 122 without disruption of the living, breeding, and/or hiding spaces 116. Discuss of food and the movement of food through the structure 102 will be discussed later.

The volume of spaces 116 can also serve as a “safe space” or a “space of solitude” for the insects, allowing them to isolate or break away from the other insects. The volume of spaces 116 can also serve as a protective enclave for the insects, allowing them to hunker in a defilade-like structure during transport of the habitat 100, etc. The columns 118 of spaces 116 can facilitate social interactions, formation of social habitats, etc. For instance, while crickets tend to be solitary creatures, social groups or mini habitats can be formed via the columnar structure—e.g., one column 118 can be “designated” for one social group while another column 118 can be for another social group.

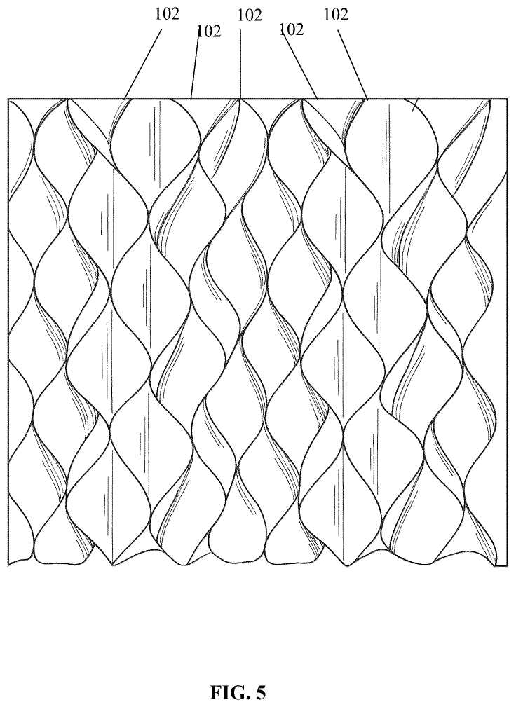

The habitat 100 can be a laminated structure 102. FIG. 5 shows a close up view of an exemplary laminated structure 102 that can be formed. For instance, the habitat 100 can be a block (e.g., a cubic formation—see FIGS. 1-2, for example) comprising plural sheets 104 that are arranged together. Each sheet 104 can be a layer or a laminate for the structure. It is contemplated for at least one of the sheets 104 to be made of cardboard (e.g., paper pulp, recycled woodchips, wood shavings, etc.); however, the sheet(s) 104 can be made of other materials such as plastic, foam, composite material, etc. Because recycled material tends to include impurities that may be hazardous to the insects, it may be preferred to have the sheet(s) 104 be made from non-recycled paper pulp.

One or more of the sheets 104 can be coated with a material to provide desired properties (e.g., rigidity, moisture resistant, etc.). For instance, the sheet(s) 104 can be coated with a cellulose. The sheet(s) 104 made from cardboard can absorb moister, breed fungi, become damp, lose structural integrity (e.g., lose rigidity), etc. Thus, coating the sheet(s) 104 with cellulose can reduce or eliminate any of these effects.

To form the laminated structure 102, a sheet 104 can be attached to or adhered to its adjacent sheet 104. It is contemplated for each sheet 104 to be attached to or adhered to its adjacent sheet 104; however, any number of sheets 104 within the structure 102 can be attached or adhered to one another, while any number of sheets 104 can be placed adjacent another sheet 104 but not adhered to it. Attaching or adhering the sheets 104 together can be accomplished by glueing the sheets 104 together (e.g., applying an adhesive to a crest 106 and/or through 108 of one sheet 104 so that when it abuts its adjacent sheet 104, the glue causes the sheets 104 to adhere to each other). The glue can be an adhesive such as, a starch glue, a dextrin glue, a polysaccharide glue, an epoxy, etc.

The laminated structure 102 can be formed into blocks (e.g., a cubic block). Dimensions of the block can be set to achieve desired design criteria, such as optimizing manufacturing costs, satisfying customer demands, optimizing environmental conditions for the type for insect, etc. In addition, a user (e.g., customer) can cut the block into desired dimensions after purchasing the structure 102. The laminated structure 102 of the habitat 100 allows for such manipulation (e.g., cutting into desired sizes and shapes) without falling apart. While a block structure is described herein, the inventive habitat 100 can be cut into any desired geometric shape to fit into an appropriately sized container (e.g., rectangular, cylindrical, spherical, triangular, etc.).

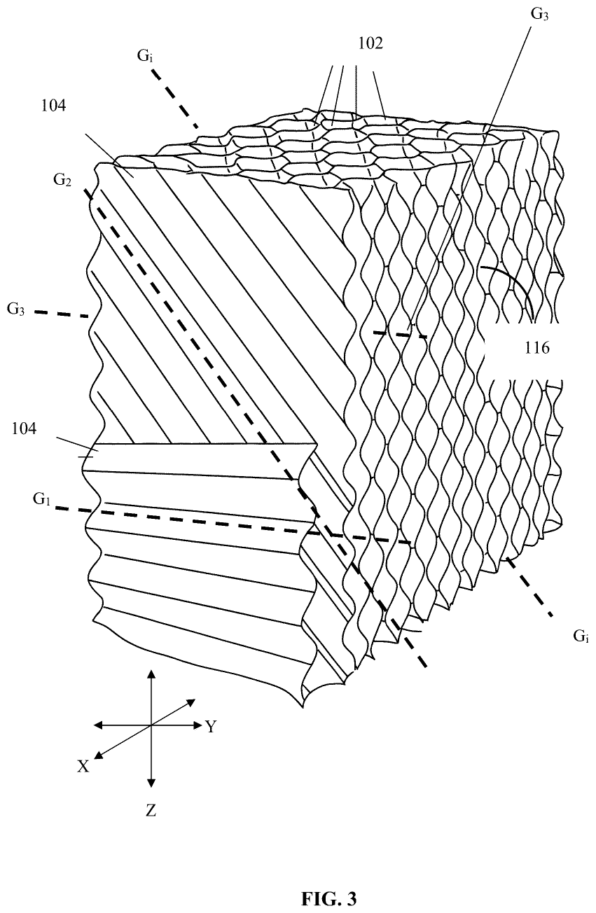

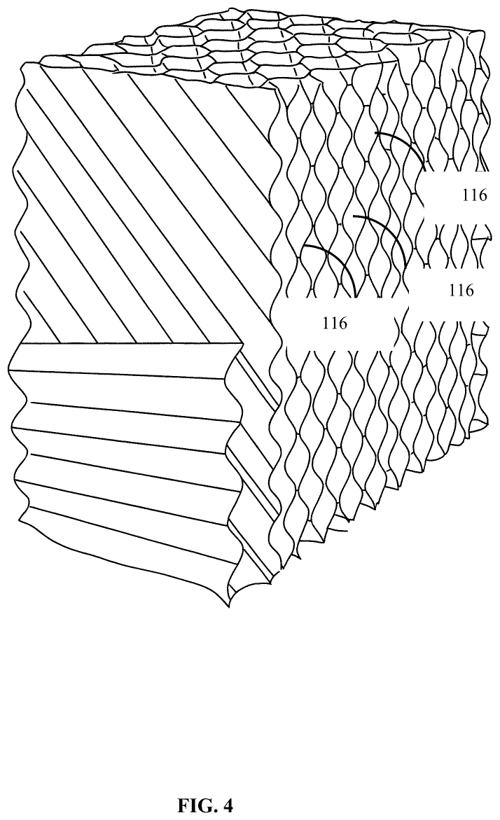

Referring to FIGS. 3-4, the laminated structure 102 can include plural sheets 104. Each sheet 104 can have a first surface 110 and a second surface 112. One or more of the sheets 104 can include an undulated profile (e.g., a corrugated profile). For instance, each sheet 104 can have a series of crests 106 and throughs 108. For each sheet 104, an apex of each crest 106 can form a ridge on the first surface 110. For each sheet 104, an apex of each ridge can form a ridge on the second surface 112. For each sheet 104, a volume of space between two adjacent crests 106 can form a groove 114 on the first surface 110. For each sheet 104, a volume of space between two adjacent throughs 108 can form a groove 114 on the second surface 112. In other words, each sheet 104 can have an undulating surface in which the first surface 110 has a plurality of grooves 114 and the second surface 112 has a plurality of grooves 114 and the positions of the grooves 114 in the first surface 110 alternates with the positions of the grooves on the second surface 112. Each groove 114 can have a longitudinal axis (generally referred to as G). As noted herein, the laminated structure 102 can include plural sheets 104. Thus, each sheet 104 can have plural grooves 114 and each groove 114 on each sheet 104 can have a longitudinal axis G. For instance, a first sheet 104 can have plural grooves 114 with each groove 114 having a longitudinal axis G₁, a second sheet 104 can have plural grooves 114 with each groove 114 having a longitudinal axis G₂, a third sheet 104 can have plural grooves 11 with each groove 114 having a longitudinal axis G₃, an i^{th} sheet 104 can have plural grooves 114 with each groove 114 having a longitudinal axis Gᵢ, etc. It is contemplated for the grooves 114 of one sheet 104 to not be parallel to the grooves 114 of an adjacent sheet 104 in the laminated structure 102. Thus, assuming the first sheet 104 is adjacent the second sheet 104 and the third sheet 104 is adjacent the second sheet 104 in the laminated structure 102, then G₁ would not be parallel to G₂ and G₂ would not be parallel to G₃, etc. As will be explained herein, this non-parallel configuration can generate plurality of spaces 116 (e.g., living spaces) that are desired within the laminated structure 102.

Each sheet 104 can be arranged in the laminated structure 102 so that a crest 106 of one sheet 104 abuts a through 108 of an adjacent sheet 104. As noted above, it is contemplated for G₁ of one sheet 104 to not be parallel to G₂ of an adjacent sheet 104. For instance, a first sheet 104 can have grooves 114 (having directions defined by G₁ of that that sheet 104). A second sheet 104 can have grooves 114 (having directions defined by G₂ of that that sheet 104). The laminated structure 102 can include the first sheet 104 being adjacent to the second sheet 104. It is contemplated for the grooves 114 of the first sheet 104 to not be parallel to the grooves 114 of the second sheet 104. As an example, if the laminated structure 102 were placed in a Cartesian coordinate system (see FIG. 3, for example), the grooves 114 of the first sheet 104 can be at a 0° angle with respect to the x-y plane (or 90° angle with respect to the z-axis), and the grooves 114 of the second sheet 104 can be at a 75° angle with respect to the x-y plane (or 15° angle with respect to the z-axis). It is contemplated for the laminate structure 102 to have the plurality of sheets 104 arranged in an alternating angle orientation. Thus, the first sheet 104 grooves 114 can be at a 0° angle, the second sheet 104 grooves 114 can be at a 75° angle, the third sheet 104 groves 114 can be at a 0° angle, the fourth sheet 102 groves 114 can be at a 75° angle, etc. This 0° angle-75° angle formation is exemplary, and it is understood that other angles can be used. It is further understood that the alternating angle orientation is also exemplary, as the angle orientations need to alternate as described above. For instance, the first sheet 104 can have a 0° angle, the second sheet 104 can have a 75° angle, the third sheet 104 can have a 35° angle, the fourth sheet 104 can have a 75° angle, the fifth sheet 104 can have a 0° angle, etc.

FIGS. 3-4 show an exemplary embodiment in which the grooves 114 of the first sheet 104 are at a 15° angle with respect to the to the y-axis and the grooves 114 of the second sheet 104 are at a 45° angle with respect to the y-axis. The laminate structure 102 in this exemplary embodiment has the plurality of sheets 104 arranged in an alternating angle orientation, and thus the first sheet 104 grooves 114 are at a 15° angle, the second sheet 104 grooves 114 are a 45° angle, the third sheet 104 groves 114 are at a 15° angle, the fourth sheet 104 groves 114 are at a 45° angle, etc. FIGS. 3-4 depict a configuration with the outermost sheet 104 removed, revealing an adjacent sheet 104 oriented at approximately 45° in an opposite direction relative to the removed sheet 104. This alternating angular orientation contributes to the formation of intersecting channels (e.g., grooves 114) that optimize both airflow and usable habitat space(s) 116.

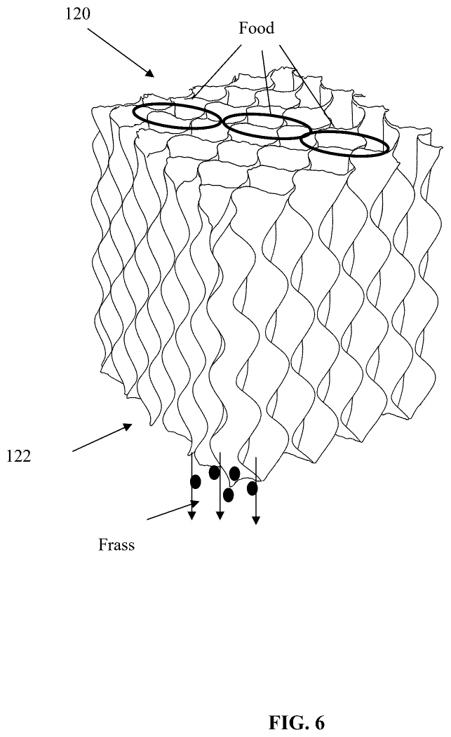

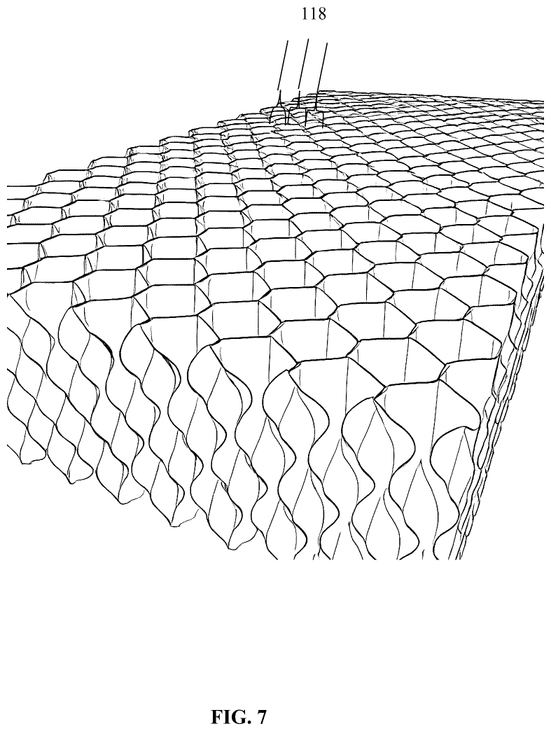

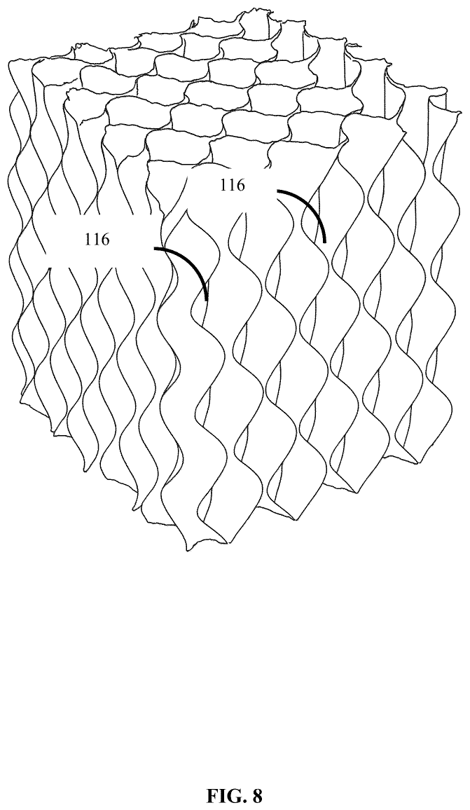

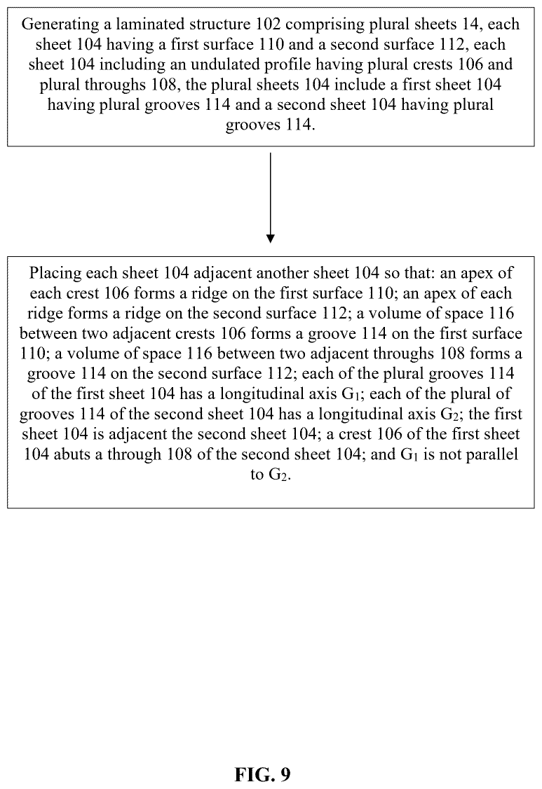

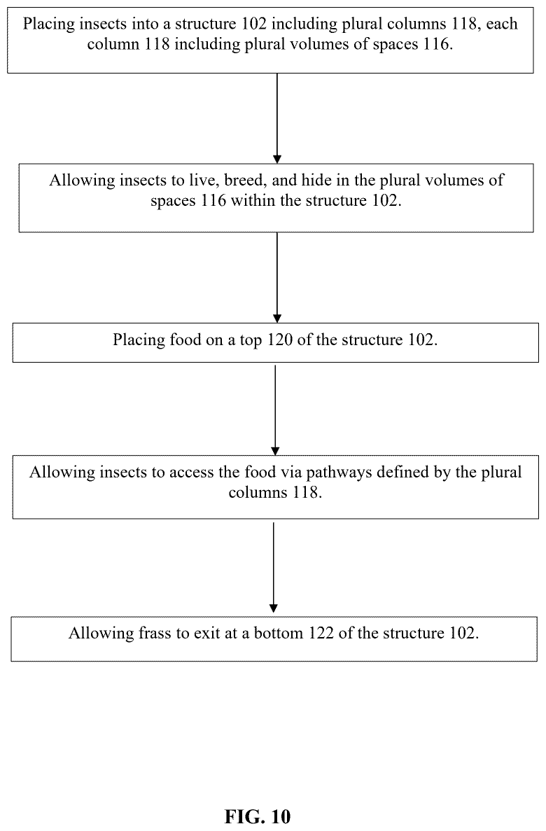

Referring to FIGS. 6-8, the volume(s) of space 116 between each sheet 104 in the laminate structure 102 can form a column 118, wherein the grooves 114 and abutment points of two adjacent sheets 104 can provide for plural living, breeding, and hiding spaces 116 within each column 118. Thus, the abutment of each sheet 104 at the crests 106 and troughs 108, the non-parallel groove orientations, and the alternating groove orientations can provide for optimum habitat conditions for the insects. For instance, each column 118 can provide multiple spaces 116 for insect movement, resting, hiding, feeding, breeding, movement, etc. In addition, food can be placed on a top 120 of the block, wherein insects can climb to the top 120 to eat or retrieve the food. Yet, frass falls down the column(s) 118 to the bottom 122 of the block, thereby reducing or eliminating food contamination. Furthermore, the volume(s) of space 116 in each column 118 provide(s) adequate air flow for ventilation, while also maintaining comfortable space(s) 118 for the insects to reduce or avoid stress. This can also provide secure space(s) 118 for the insects to reduce or eliminate disturbance if/when the block is transported.

In addition, a user can cut holes, tunnels, channels, etc. into the block to provide for additional/desired ventilation, movement pathways, desired internal structures, etc.

While exemplary embodiments may describe each sheet 104 having an undulated profile, it is understood that any number of sheets can have an undulated profile. Furthermore, while exemplary embodiments may describe and illustrate the undulations being sinusoidal, other formations can be used (e.g., triangular shapes, square shapes, etc.). Moreover, while exemplary embodiments may describe and illustrate each crest 106 of one sheet 104 abutting a through 108 of an adjacent sheet 104, other arrangements can be made.

Embodiments can relate to a method of forming a habitat 100 for insects. The method can involve generating a laminated structure 102 having plural sheets 104. Each sheet 104 can have a first surface 110 and a second surface 112. Each sheet 104 can include an undulated profile having plural crests 106 and plural throughs 108. The plural sheets 104 include a first sheet 104 having plural grooves 114, a second sheet 104 having plural grooves 114, a third sheet 104 having plural grooves 114, etc. The method can involve placing each sheet 104 adjacent another sheet 104 so that an apex of each crest 106 forms a ridge on the first surface 110 of a sheet 104, an apex of each ridge forms a ridge on the second surface 112 of a sheet 104; a volume of space 116 between two adjacent crests 106 of a sheet 104 forms a groove 114 on the first surface 110 of that sheet 104, and a volume of space 116 between two adjacent throughs 108 of a sheet 104 forms a groove 114 on the second surface 112 of that sheet 104. Each of the plural grooves 114 of the first sheet 104 has a longitudinal axis G₁, each of the plural of grooves 114 of the second sheet 104 has a longitudinal axis G₂. A crest 106 of the first sheet 104 abuts a through 108 of the second sheet 104. G₁ is not parallel to G₂. This same configuration can be applied to the third sheet 104 with respect to the second sheet 104, the fourth sheet 104 with respect to the third sheet 104, etc.

Embodiments can relate to a method of maintaining a habitat 100 for insects. The method can involve placing insects into a structure 102 including plural columns 118. Each column 118 can include plural volumes of spaces 116. The method can involve allowing insects to live, breed, and hide in the plural volumes of spaces 116 within the structure 102. The method can involve placing food on a top 120 of the structure 102. The method can involve allowing insects to access the food via pathways defined by the plural columns 118. The insects can bring the food back to their respective living space 116 to hoard the food (e.g., hide food from other insects), eat the food, store the food, share the food, etc. The insect can consume the food at its leisure. After consuming the food, the insect can defecate and allow/force the fuss from the living space 116 so that it exits the structure 102 via the column 118 to which the living space 116 is associated—e.g., the fuss travels down the pathway of the column 118 to exist an egress formed at the bottom 122.

The method can involve cutting the structure 102 into at least one block or sub-block. In addition, a user can cut holes, tunnels, channels, etc. into the block or sub-block to provide for additional/desired ventilation, movement pathways, desired internal structures, etc. For instance, FIG. 2 shows an exemplary embodiment of a modular block configured in a generally cubic form, approximately 3 inches by 3 inches by 3 inches in size. Alternative dimensions may be employed to accommodate different container volumes or insect species. As can be appreciated, the outermost sheet 104 on the left-hand side is oriented at an angle of approximately 45° relative to the horizontal, extending from the bottom 122 toward the top 120, facilitating multi-directional access and structural stability.

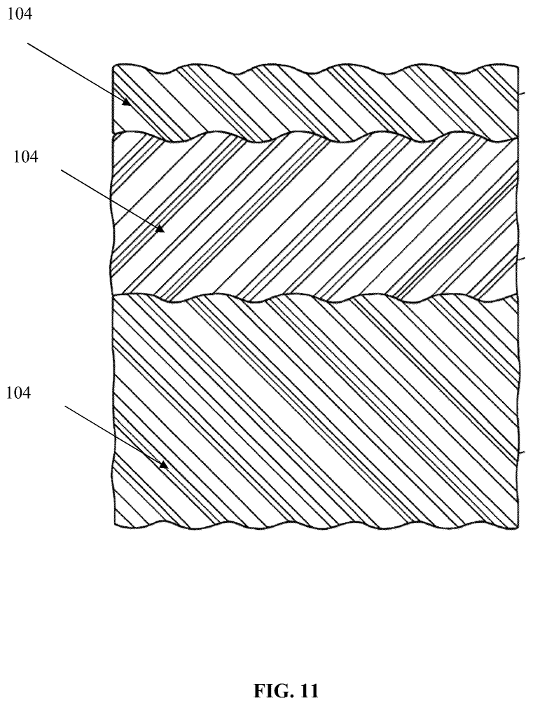

FIG. 11 depicts a side view showing sequential layering of sheets 104. In this exemplary embodiment, a first sheet 104 extends from right to left in a bottom-to-top orientation. The immediately adjacent second sheet 104 extends from left to right in a bottom-to-top orientation, opposing the first sheet 104. A third sheet 104 resumes the orientation of the first sheet 104, and this alternating sequence continues through the habitat 100. This arrangement enhances rigidity and creates an interlaced pathway system for insect movement.

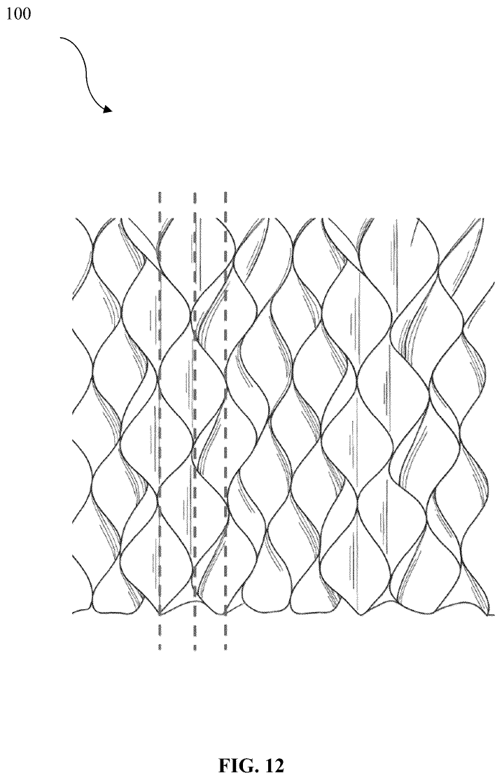

FIG. 12 depicts a front view of an exemplary habitat 100 in which interleaving sheets 104 form discrete, interconnected cell compartments (e.g., spaces 116). The resulting wavy cell compartment pattern facilitates entry and exit of insects while minimizing escape risk during handling. Dashed lines in the figure indicate the composite cell size formed between opposing sheet 104 orientations, which may be optimized for species-specific behavior and size.

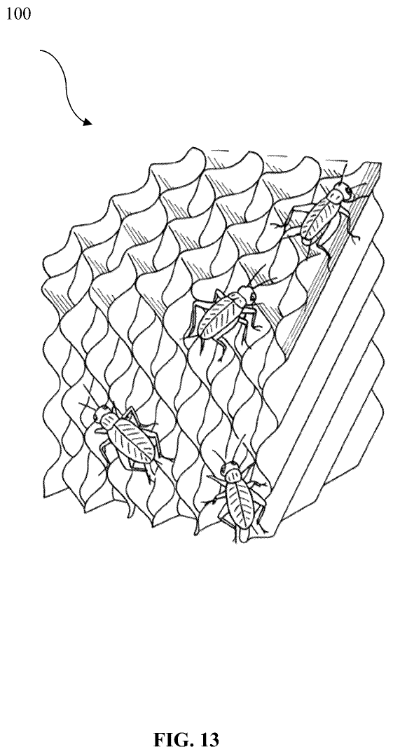

FIG. 13 depicts an example of live insect interaction with an embodiment of the habitat 100. Crickets are shown entering, exiting, and traversing over the surfaces. The spatial configuration of the habitat 100 supports high-density occupancy while maintaining ventilation and accessibility for feeding and maintenance.

### EXAMPLES

The following describes exemplary implementations and test results of embodiments of the habitat. It is understood that the following are exemplary only and that embodiments disclosed herein are not limited to the examples that follow.

An objective of the inventive habitat 100 can be to provide the best possible habitat structure 102 for crickets during captivity. This can be achieved by a series of improvements, such as offering adequate hiding spaces 116 for molting crickets to reduce cannibalism, increasing the overall surface area for perching (hanging) to minimize stress, and incorporating a stable structure 102 to separate food from frass, thereby reducing contamination and other biohazard risks for the insects.

Another objective can be to mitigate the adverse effects associated with transporting crickets by air and road. Excessive movement and shaking during transit can stress the insects, while extreme temperatures during summer and winter can cause premature deaths. The inventive habitat 100 can address these issues by improving the insects'survival rates and overall well-being through its carefully designed structure 102 that minimizes empty spaces.

The inventive habitat 100 can be designed to function as an ideal structure 102 for packaging and displaying crickets on retail shelves. Live feed sales for exotic pets have become a growing market opportunity, but current packaging and housing materials often fail to ensure cricket survivability, leading to premature deaths and financial losses for retailers.

Referring to FIG. 1, the inventive habitat 100 can be configured as a rigid structure 102 made from coated cellulose paper sheets 104, fluted into an (non-specific) angle and alternating the cellulose sheets 104 with different (non-specific) angles, creating a crossing two-way opening path 114. The resulting concave shapes 114 can provide hiding spaces 116 for molting crickets, help maintain ideal temperatures within the habitat, and reduce the empty spaces where crickets could be unwantedly shaken around.

Referring to FIGS. 1-2, the inventive habitat 100 can be a block-shaped (cube or rectangular prism) structure 102 configured to serve as a housing system (condominium) for insect colonies. The overlapping sheets 104 with two different angles can create small apertures 116 that crickets naturally find appealing, as they prefer dark and tight spaces 116 where they are safe. The insulating properties of cardboard can help maintain a more regulated temperature, which is ideal for ectothermic insects.

Beneficial Features:
Material Application: the inventive habitat 100 can utilize a coated cellulose paper, an innovation in insect rearing materials, offering enhanced durability and added functionalities like moisture resistance and antimicrobial properties. This represents a significant advancement over traditional untreated cardboard habitats.
Unique Structural Design: The cross-cell architecture 102 can improve air circulation and temperature regulation within the habitat 100. This structure 102 can be tailored specifically to meet the behavioral and physiological needs of crickets, marking a departure from conventional habitat designs.
Customization and Modularity: The inventive habitat's 100 adaptable design can allow for customization in size, shape, and material thickness, catering to various insect species and rearing environments. This versatility can expand its application beyond traditional cricket rearing methods.
Scalability and Automation Potential: Designed with scalability in mind, the inventive habitat 100 can be seamlessly integrated into automated rearing systems, supporting both small-scale operations and large commercial farms. This innovation facilitates efficient production while maintaining the integrity of the insect habitat.
Environmental and Economic Efficiency: The use of renewable, recyclable materials in the inventive habitat 100 not only promotes environmental sustainability, but can also introduce cost efficiencies in production and operation. This focus on sustainability is a novel approach in the field of insect rearing habitats.
Enhanced Biosecurity and Sanitation: The structure's 102 design can include a feature that separates food from frass, reducing biohazard risks and maintaining sanitation within the habitat 100. This is a significant innovation in ensuring a safe and clean environment for crickets.
Maximizes Total Perching (Hanging) Surface: The inventive habitat 100 can maximize the available hanging area within a volumetric container, allowing for a higher density of crickets in a given space, thus improving rearing efficiency.
Improves Hiding Space for Molting Crickets and Reduces Cannibalism: Molting crickets are vulnerable to cannibalism due to their soft exoskeleton. The inventive habitat 100 can provide ample hiding spaces 116 to protect them during this critical period, reducing losses and colony reduction.
Minimizes Empty Space and Reduces Impact During Transport: The closed-cell design 102 of the inventive habitat 100 can minimize empty space between sheets, cushioning and reducing the impact of package movement during transport. This reduces stress and physical damage to the insects.
Environmental and Temperature Benefits: The biodegradable and insulating properties of the cellulose material can help maintain a stable temperature, benefiting the crickets and minimizing environmental impact.
Multi-functional Structure: The solid block structure can allow for the placement of feed on top 120, while frass falls to the bottom 122 of the container 102. This can reduce potential biohazard risks and enhances the habitat environment for crickets.
Ease of Integration: The inventive habitat can be adaptable to a wide variety of packaging containers, such as cardboard boxes, plastic jars, and even flexible bags and meshes.
Air Circulation and Temperature Regulation: The cross-cell design can promote air circulation in two directions, effectively regulating the temperature within the container 102.
Simplified Assembly: The one-piece block design can reduce labor and complexity in packaging assembly, unlike other systems that require multiple components (e.g., egg crates, cardboard sheets, grids).

The inventive habitat 100 is an efficient structure that can serve as an ideal solution for mass rearing, long-distance transportation, and retail display of crickets. Its multifunctionality addresses many of the challenges associated with current materials like egg crates and grid-shaped cardboard, making it a game-changer across various industries.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment.

Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the systems, apparatuses, and methods of using and making the same disclosed herein have been discussed and illustrated, it is to be distinctly understood that the invention is not limited thereto but may be otherwise variously embodied and practiced within the scope of the following claims.

What is claimed is:

1. A habitat for insects, comprising:
plural sheets;
wherein:
one sheet is arranged adjacent to another sheet;
each sheet has a surface ornamentation to generate plural abutment points and plural volumes of spaces between two adjacent sheets;
each volume of space provides a living, breeding, and/or hiding space for an insect;
the plural sheets form a structure having a top and a bottom;
the plural volume of spaces forms a column defining a pathway from the top to the bottom;
the pathway facilitating passage of frass within the column in a direction from the top to the bottom.

2. The habitat of claim 1, wherein:
the column and plurality of spaces are configured to facilitate providing plural living, breeding, and/or hiding spaces for an insect while also facilitating passage of frass within the column in a direction from the top to the bottom without disruption of the living, breeding, and/or hiding spaces.

3. The habitat of claim 1, wherein:
the plural sheets form plural columns, each column having plural volume of spaces.

4. The habitat of claim 1, wherein:
at least one sheet is made of cardboard.

5. The habitat of claim 1, wherein:
each sheet is made of cardboard.

6. The habitat of claim 1, wherein:
at least one sheet is made of non-recycled cardboard.

7. The habitat of claim 1, wherein:
each sheet is made of non-recycled cardboard.

8. The habitat of claim 1, wherein:
at least one sheet is made of cardboard coated with cellulose.

9. The habitat of claim 1, wherein:
each sheet is made of cardboard coated with cellulose.

10. The habitat of claim 1, wherein:
at least one sheet is adhered to an adjacent sheet via an adhesive.

11. The habitat of claim 1, wherein:
each sheet is adhered to an adjacent sheet via an adhesive.

12. A habitat for insects, comprising:
a laminated structure comprising plural sheets;
wherein:
each sheet has a first surface and a second surface;
each sheet includes an undulated profile having plural crests and plural throughs;
for each sheet:
an apex of each crest forms a ridge on the first surface;
an apex of each ridge forms a ridge on the second surface;
a volume of space between two adjacent crests forms a groove on the first surface;
a volume of space between two adjacent throughs forms a groove on the second surface;
the plural sheets include a first sheet having plural grooves and a second sheet having plural grooves;
each of the plural grooves of the first sheet has a longitudinal axis G₁;
each of the plural of grooves of the second sheet has a longitudinal axis G₂;
the plural sheets are arranged in the laminated structure so that:
the first sheet is adjacent the second sheet;
a crest of the first sheet abuts a through of the second sheet; and
G₁ is not parallel to G₂.

13. A method of forming a habitat for insects, the method comprising:
generating a laminated structure comprising plural sheets, each sheet having a first surface and a second surface, each sheet including an undulated profile having plural crests and plural throughs, the plural sheets include a first sheet having plural grooves and a second sheet having plural grooves;
placing each sheet adjacent another sheet so that:
an apex of each crest forms a ridge on the first surface;
an apex of each ridge forms a ridge on the second surface;
a volume of space between two adjacent crests forms a groove on the first surface;
a volume of space between two adjacent throughs forms a groove on the second surface;
each of the plural grooves of the first sheet has a longitudinal axis G₁;
each of the plural of grooves of the second sheet has a longitudinal axis G₂;
the first sheet is adjacent the second sheet;
a crest of the first sheet abuts a through of the second sheet; and
G₁ is not parallel to G₂.

14. The method of claim 13, comprising:
forming each sheet from cardboard.

15. The method of claim 13, comprising:
forming each sheet from non-recycled cardboard.

16. The method of claim 13, comprising:
coating each sheet with cellulose.

17. A method of maintaining a habitat for insects, the method comprising:
placing insects into a structure including plural columns, each column including plural volumes of spaces;
allowing insects to live, breed, and hide in the plural volumes of spaces within the structure;
placing food on a top of the structure;
allowing insects to access the food via pathways defined by the plural columns; and
allowing frass to exit at a bottom of the structure.

18. The method of claim 17, comprising:
cutting the structure into at least one block or sub-block.
